(19) United States
(12) Patent Application Publication
(10) Pub. No.: US 2023/0413785 A1
(43) Pub. Date: Dec. 28, 2023

(54) METHOD FOR MANAGING HEALTH CONDITION OF CALF, SYSTEM FOR MANAGING HEALTH CONDITION OF CALF, APPARATUS FOR MANAGING HEALTH CONDITION OF CALF, AND GARMENT FOR MANAGING HEALTH CONDITION OF CALF

(71) Applicant: TOYOBO CO., LTD., Osaka (JP)

(72) Inventor: Yoko KOMATSU, Shiga (JP)

(73) Assignee: TOYOBO CO., LTD., Osaka (JP)

(21) Appl. No.: 18/037,358

(22) PCT Filed: Nov. 9, 2021
(86) PCT No.: PCT/JP2021/041178

§ 371 Date: May 17, 2023

(30) Foreign Application Priority Data
Nov. 20, 2020 (JP) 2020-193645

(51) Int. Cl. A01K 29/00; A61B 5/0205; A61B 5/11; A61B 5/352

(52) U.S. Cl. CPC A01K 29/005; A61B 5/02055; A61B 5/0245; A61B 5/352; A61B 5/11

(57) ABSTRACT

An object of the present invention is to provide a method for easily managing a health condition of a calf. The method for managing a health condition of a calf, including: calculating at least one of an average value of beat intervals, a variation in beat intervals, a beat number, HF, or LF/HF in the first resting period and the second resting period in which the acceleration is the constant M or less is satisfied; issuing a warning for notifying abnormality of a body temperature of the calf when a magnitude relationship of at least one of formulae (1) to (5) below; and raising or lowering the body temperature of the calf:

the average value of beat intervals in the first resting period>the average value of beat intervals in the second resting period (1)

the beat number in the first resting period<the beat number in the second resting period (2)

the variation in beat intervals in the first resting period>the variation in beat intervals in the second resting period (3)

the HF in the first resting period>the HF in the second resting period (4)

the LF/HF in the first resting period<the LF/HF in the second resting period (5).

### TECHNICAL FIELD

The present invention relates to a method for managing a health condition of a calf, a system for managing a health condition of a calf, an apparatus for managing a health condition of a calf, and a garment for managing a health condition of a calf.

### BACKGROUND ART

In recent years, apparatuses and systems for measuring biological information of animals have attracted attention. For example, Patent Document 1 discloses a childbirth monitoring apparatus including a heart rate meter that measures a heart rate of an animal mother body whose childbirth is to be monitored, a circuit that discriminates, from the heart rate detected by the heart rate meter, a temporary decrease in a heart rate exceeding a fluctuation range of the heart rate in a normal time before childbirth, and a communication apparatus and/or an alarm apparatus that notifies an immediately-before-childbirth signal due to the decrease in the heart rate output from the discrimination circuit.

### RELATED ART DOCUMENT

### Patent Document

Patent Document 1: JP-A-2008-011916

### SUMMARY OF THE INVENTION

### Problems to be Solved by the Invention

Conventionally, particularly in calves after birth, an immune function has easily deteriorated and a mortality rate has been high. In addition, it has been difficult to manage a health condition of a calf since the calf instinctively tries to hide a poor physical condition. The present invention has been made in view of the above situations, and an object of the present invention is to provide a method for easily managing a health condition of a calf. Another object of the present invention is to provide a system, an apparatus, and a garment for easily managing a health condition of a calf.

### Solution to the Problems

A method for managing a health condition of a calf according to an embodiment of the present invention capable of solving the above problem is as described in [1] below.

[1] A method for managing a health condition of a calf, including:
measuring beat intervals and acceleration associated with movement;
specifying a first resting period in which the acceleration is a constant M or less;
specifying a second resting period that is a period having a starting point at a time point after an end point of the first resting period and in which the acceleration is the constant M or less;
calculating at least one of an average value of beat intervals, a variation in beat intervals, a beat number, H F, or LF/HF in the first resting period;
calculating at least one of an average value of beat intervals, a variation in beat intervals, a beat number, HF, or LF/HF in the second resting period;
issuing a warning for notifying abnormality of a body temperature of the calf when a magnitude relationship of at least one of formulae (1) to (5) below is satisfied; and
raising or lowering the body temperature of the calf:

the average value of beat intervals in the first resting period>the average value of beat intervals in the second resting period (1)

the beat number in the first resting period<the beat number in the second resting period (2)

the variation in beat intervals in the first resting period>the variation in beat intervals in the second resting period (3)

the HF in the first resting period>the HF in the second resting period (4)

the LF/HF in the first resting period<the LF/HF in the second resting period (5)
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.

The abnormality of the body temperature of the calf can be determined on the basis of the magnitude relationships of formulae (1) to (5) above. By raising or lowering the body temperature of the calf after the warning of the abnormality, it is possible to prevent deterioration of a poor physical condition of the calf. A preferable aspect of the method for managing a health condition of a calf is any one of [2] to [8], and [1a] to [1c] below.

[2] The method for managing a health condition of a calf according to [1], wherein the body temperature of the calf is raised by using a heat retaining garment, heating equipment, or a hot-water supply machine.

[3] The method for managing a health condition of a calf according to [1] or [2], wherein the calf is cattle under nine months of age.

[4] The method for managing a health condition of a calf according to any one of [1] to [3], wherein each of the average values of beat intervals is an average value of RRIs each of which is an interval between an R wave and an R wave in an electrocardiographic signal.

[5] The method for managing a health condition of a calf according to any one of [1] to [4], wherein the beat number in the first resting period and the beat number in the second resting period are each a heart rate or a pulse rate.

[6] The method for managing a health condition of a calf according to any one of [1] to [5], wherein each of the variations in beat intervals is at least one of SDNN, RMSSD, CVRR, NN50, or pNN50,
provided that, the SDNN is a standard deviation of an RRI that is an interval between an R wave and an R wave in an electrocardiographic signal,
the RMSSD is a square root of an average value of squares of differences between consecutive adjacent RRIs,
the CVRR is a value obtained by dividing a value of the SDNN by an average value of RRIs and multiplying the obtained value by 100,
the NN50 is a value indicating a total number of times in which differences between the consecutive adjacent RRIs exceed 50 ms, and
the pNN50 is a value indicating a ratio of heartbeats in which the differences between the consecutive adjacent RRIs exceed 50 ms.

[7] The method for managing a health condition of a calf according to any one of [1] to [6], wherein the acceleration is expressed by a formula below:

[Mathematical formula 1]

Acceleration A=\sqrt{x²+y²+z²}−1 (I)
wherein x, y, and z are X-axis, Y-axis, and Z-axis acceleration obtained from an accelerometer attached to a body of the calf.

[8] The method for managing a health condition of a calf according to any one of [1] to [7], wherein the beat intervals are measured using a biological information measuring garment.
[1a] The method for managing a health condition of a calf according to any one of [1] to [8], wherein
in calculating each of the first resting period and the second resting period, at least one of the average value of beat intervals, the variation in beat intervals, or the beat number is calculated, and
in issuing the warning, the warning is issued when the magnitude relationship of at least one of formulae (1) to (3) above is satisfied.
[1b] The method for managing a health condition of a calf according to any one of [1] to [8], wherein
in calculating each of the first resting period and the second resting period, at least one of the average value of beat intervals or the beat number is calculated, and
in issuing the warning, the warning is issued when the magnitude relationship of at least one of formula (1) or formula (2) above is satisfied.
[1c] The method for managing a health condition of a calf according to any one of [1] to [8], wherein
in calculating each of the first resting period and the second resting period, the average value of beat intervals is calculated, and
in issuing the warning, the warning is issued when the magnitude relationship of formula (1) above is satisfied.

A system for managing a health condition of a calf according to an embodiment of the present invention that has been able to solve the above problem is as described in [9], and a preferable aspect is as described in any one of [10] to [14], and [9a] to [9c] below.

[9] A system for managing a health condition of a calf, including:
a measurement unit that measures beat intervals and acceleration associated with movement;
a specification unit that specifies a first resting period in which the acceleration is a constant M or less, and specifies a second resting period that is a period having a starting point at a time point after an end point of the first resting period and in which the acceleration is the constant M or less;
a calculation unit that calculates at least one of an average value of beat intervals, a variation in beat intervals, a beat number, HF, or LF/HF in the first resting period, and calculates at least one of an average value of beat intervals, a variation in beat intervals, a beat number, H F, or LF/HF in the second resting period;
a determination unit that generates a warning signal when a magnitude relationship of at least one of formulae (1) to (5) below is satisfied; and
a warning unit that issues a waring for notifying abnormality of a body temperature of the calf from the warning signal:

the average value of beat intervals in the first resting period>the average value of beat intervals in the second resting period (1)

the beat number in the first resting period<the beat number in the second resting period (2)

the variation in beat intervals in the first resting period>the variation in beat intervals in the second resting period (3)

the HF in the first resting period>the HF in the second resting period (4)

the LF/HF in the first resting period<the LF/HF in the second resting period (5)
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.

[10] The system for managing a health condition of a calf according to [9], including a thermoregulation unit that raises or lowers the body temperature of the calf.

[11] The system for managing a health condition of a calf according to [9] or [10], wherein each of the average values of beat intervals is an average value of RRIs each of which is an interval between an R wave and an R wave in an electrocardiographic signal.

[12] The system for managing a health condition of a calf according to any one of [9] to [11], wherein the beat number in the first resting period and the beat number in the second resting period are each a heart rate or a pulse rate.

[13] The system for managing a health condition of a calf according to any one of [9] to [12], wherein each of the variations in beat intervals is at least one of SDNN, RMSSD, CVRR, NN50, or pNN50,
provided that, the SDNN is a standard deviation of an RRI that is an interval between an R wave and an R wave in an electrocardiographic signal,
the RMSSD is a square root of an average value of squares of differences between consecutive adjacent RRIs,
the CVRR is a value obtained by dividing a value of the SDNN by an average value of RRIs and multiplying the obtained value by 100,
the NN50 is a value indicating a total number of times in which the differences between the consecutive adjacent RRIs exceed 50 ms, and
the pNN50 is a value indicating a ratio of heartbeats in which the differences between the consecutive adjacent RRIs exceed 50 ms.

[14] The system for managing a health condition of a calf according to any one of [9] to [13], wherein the acceleration is expressed by a formula below:

[Mathematical formula 2]

Acceleration A=\sqrt{x²+y²+z²}−1 (I)
wherein x, y, and z are X-axis, Y-axis, and Z-axis acceleration obtained from an accelerometer attached to a body of the calf.

[9a] The system for managing a health condition of a calf according to any one of [9] to [14], wherein
the calculation unit calculates at least one of the average value of beat intervals, the variation in beat intervals, or the beat number in each of the first resting period and the second resting period, and
the determination unit issues the warning signal when the magnitude relationship of at least one of formulae (1) to (3) above is satisfied.
[9b] The system for managing a health condition of a calf according to any one of [9] to [14], wherein
the calculation unit calculates at least one of the average value of beat intervals or the beat number in each of the first resting period and the second resting period, and
the determination unit issues the warning signal when the magnitude relationship of at least one of formula (1) or formula (2) above is satisfied.
[9c] The system for managing a health condition of a calf according to any one of [9] to [14], wherein
the calculation unit calculates the average value of beat intervals in each of the first resting period and the second resting period, and
the determination unit issues the warning signal when the magnitude relationship of formula (1) above is satisfied.

A garment for managing a health condition of a calf and an apparatus for managing a health condition of a calf according to an embodiment of the present invention that have been able to solve the above problem is as described in [15] and [16] and preferable aspects are as described in any one of [16a] to [16c] below.
[15] A garment including at least a part of the system for managing a health condition of a calf according to any one of [9] to [14] and [9a] to [9c].
[16] An apparatus for managing a health condition of a calf, including:
a measurement unit that measures beat intervals and acceleration associated with movement;
a specification unit that specifies a first resting period in which the acceleration is a constant M or less, and specifies a second resting period that is a period having a starting point at a time point after an end point of the first resting period and in which the acceleration is the constant M or less;
a calculation unit that calculates at least one of an average value of beat intervals, a variation in beat intervals, a beat number, HF, or LF/HF in the first resting period, and calculates at least one of an average value of beat intervals, a variation in beat intervals, a beat number, H F, or LF/HF in the second resting period; and
a determination unit that generates a warning signal when a magnitude relationship of at least one of formulae (1) to (5) below is satisfied:

the average value of beat intervals in the first resting period>the average value of beat intervals in the second resting period (1)

the beat number in the first resting period<the beat number in the second resting period (2)

the variation in beat intervals in the first resting period>the variation in beat intervals in the second resting period (3)

the HF in the first resting period>the HF in the second resting period (4)

the LF/HF in the first resting period<the LF/HF in the second resting period (5)
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.
[16a] The apparatus for managing a health condition of a calf according to [16], wherein
the calculation unit calculates at least one of the average value of beat intervals, the variation in beat intervals, or the beat number in each of the first resting period and the second resting period, and
the determination unit issues the warning signal when the magnitude relationship of at least one of formulae (1) to (3) above is satisfied.
[16b] The apparatus for managing a health condition of a calf according to [16], wherein
the calculation unit calculates at least one of the average value of beat intervals or the beat number in each of the first resting period and the second resting period, and
the determination unit issues the warning signal when the magnitude relationship of at least one of formula (1) or formula (2) above is satisfied.
[16c] The apparatus for managing a health condition of a calf according to [16], wherein
the calculation unit calculates the average value of beat intervals in each of the first resting period and the second resting period, and
the determination unit issues the warning signal when the magnitude relationship of formula (1) above is satisfied.

### Effects of the Invention

According to the present invention, with the above configuration, it is possible to provide a method for easily managing a health condition of a calf. In addition, it is possible to provide a system, an apparatus, and a garment for easily managing a health condition of a calf.

### BRIEF DESCRIPTION OF THE DRAWINGS

FIG. 1 is an explanatory diagram of power spectrum integration.

FIG. 2 is a block diagram illustrating a configuration of a system for managing a health condition of a calf according to a first embodiment.

FIG. 3 is a block diagram illustrating a configuration of a system for managing a health condition of a calf according to a second embodiment.

FIG. 4 is a plan view of a skin side of a biological information measuring garment.

FIG. 5 is a diagram illustrating a relationship between acceleration A measured by the system for managing a health condition of a calf of example 1, and a measurement time.

FIG. 6 is a diagram illustrating a relationship between an RRI measured by the system for managing a health condition of a calf of example 1, and a measurement time.

### MODE FOR CARRYING OUT THE INVENTION

A method for managing a health condition of a calf according to an embodiment of the present invention includes:
measuring beat intervals and acceleration associated with movement; specifying a first resting period in which the acceleration is a constant M or less; specifying a second resting period that is a period having a starting point at a time point after an end point of the first resting period and in which the acceleration is the constant M or less; calculating at least one of an average value of beat intervals, a variation in beat intervals, a beat number, HF, or LF/HF in the first resting period;
calculating at least one of an average value of beat intervals, a variation in beat intervals, a beat number, HF, or LF/HF in the second resting period;
issuing a warning for notifying abnormality of a body temperature of the calf when a magnitude relationship of at least one of formulae (1) to (5) below is satisfied; and
raising or lowering the body temperature of the calf,

the average value of beat intervals in the first resting period>the average value of beat intervals in the second resting period (1)

the beat number in the first resting period<the beat number in the second resting period (2)

the variation in beat intervals in the first resting period>the variation in beat intervals in the second resting period (3)

the HF in the first resting period>the HF in the second resting period (4)

the LF/HF in the first resting period<the LF/HF in the second resting period (5)
provided that, each LF is a value obtained by performing definite integration of a power spectrum from frequencies L_{f1} to L_{f2}, the power spectrum being obtained by including performing frequency spectrum conversion on beat intervals, and each HF is a value obtained by performing definite integration of the power spectrum from frequencies H_{f1} to H_{f2}, where H_{f1}>L_{f1} and H_{f2}>L_{f2} are satisfied.

Since the calf has a thin subcutaneous fat and a body surface of the calf is easily affected by a change in an outside air temperature, there is a possibility that an abnormal change in a core temperature cannot be detected by body temperature measurement using a body temperature sensor or the like. On the other hand, in the method for managing a health condition of a calf, it is possible to determine whether or not at least one of the average value of beat intervals, the variation in beat intervals, the beat number, the HF, or LF/HF (hereinafter, it may be simply referred to as value relating to heartbeat), which closely relates to the body temperature, satisfies one of the magnitude relationships of formulae (1) to (5) above in each of the resting periods of the calf, thereby determining occurrence of body temperature abnormality of the calf. As a result, the health condition of the calf can be easily managed, and a burden on an owner of the calf such as a livestock farmer and a dairyman can be reduced. Furthermore, by specifying the periods in which the acceleration associated with the movement of the calf is the constant M or less as the resting periods and obtaining the values relating to the heartbeat in the resting periods, noise associated with the movement of the calf can be removed, and the occurrence of the body temperature abnormality of the calf can be easily determined. Furthermore, by raising or lowering the body temperature of the calf after the warning of the abnormality, it is possible to prevent deterioration of a poor physical condition of the calf. Each of the steps will be described in detail below.

<Measuring Beat Intervals and Acceleration Associated with Movement>

As each of the beat intervals, an R-R interval that is an interval between an R wave and an R wave in an electrocardiographic signal, an interval between a pulse wave and a pulse wave, or the like may be used. Among them, the R-R interval (RRI) is preferable. The RRI indicates an interval between an R wave and a next R wave of an electrocardiogram in which an electrical signal associated with beats of a heart is recorded in time series. Since a peak appears clearly in the R wave, erroneous recognition of a peak position hardly occurs, and measurement accuracy of the beat becomes high.

The beat intervals can be measured by an electrocardiograph that measures a heartbeat, a heartbeat sensor, or the like. These may have a beat interval measuring instrument such as electrodes or a stretchable capacitor. The electrodes are preferably fixed to a skin-side surface of clothing fabric of a garment. Examples of such stretchable electrodes include electrodes described in JP-A-2020-100903. Examples of the stretchable capacitor include a capacitor element described in JP-A-2019-072048. The electrocardiograph, the heartbeat sensor, and the like are preferably disposed on a chest and/or an abdomen of the calf, and more preferably disposed on the chest.

The beat intervals may be measured using a pulse wave sensor. The pulse wave sensor, the electrocardiograph, the heartbeat sensor, and the like can be used in combination. In a case where a pulse is measured using the pulse wave sensor, near-infrared rays having a wavelength of 700 nm to 1200 nm are irradiated to an earlobe or the like of the calf, and a reflection amount of the near-infrared rays can be measured in contact or in non-contact.

The acceleration can be measured by an accelerometer or the like. Examples of the accelerometer include a uniaxial accelerometer, a biaxial accelerometer, and a triaxial accelerometer. Among them, the triaxial accelerometer is preferable. Examples of the accelerometer include myBeat manufactured by UNION TOOL Co. The accelerometer or the like is preferably disposed on the chest and/or the abdomen of the calf, and more preferably disposed on the chest.

The acceleration (hereinafter, the acceleration may be referred to as acceleration A) associated with the movement is correlated with an activity amount of the calf,

and can be used as a criterion for determining a resting state of the calf. The acceleration A is preferably a synthetic value of the acceleration of the calf and a gravitational acceleration acting on the calf, and the synthetic value may be represented by a ratio to a gravitational acceleration g (=9.8 m/As) (unit: a dimensionless amount). Specifically, as expressed by formula (I) below, the acceleration A is more preferably a value obtained by subtracting (g/g)=1 from a square root of a sum of squares of the acceleration x, y, and z in X-axis, Y-axis, and Z-axis directions, which are acceleration measured using a triaxial accelerometer, in which (g/g)=1 is the gravitational acceleration g (=9.8 m/s²) acting on the calf (here, the unit g represents a magnitude of the gravitational acceleration):

[Mathematical formula 3]

Acceleration A=\sqrt{x²+y²+z²}−1 (I)
wherein x, y, and z are X-axis, Y-axis, and Z-axis acceleration obtained from the accelerometer attached to the body of the calf.

The acceleration A may be a square root of a sum of squares of the acceleration x, y on the X-axis and the Y-axis, which are acceleration of the calf measured using a biaxial accelerometer, and may be further represented as a ratio to the gravitational acceleration g (=9.8 m/s²) (unit: the dimensionless amount). In this case, the biaxial accelerometer is preferably attached to the calf such that a measurement direction is perpendicular to a gravity direction.

In measuring the beat intervals and the acceleration associated with the movement, it is not always necessary to simultaneously measure the beat intervals and the acceleration associated with the movement. For example, the acceleration associated with the movement may be measured by simultaneously measuring the beat intervals and the acceleration, and then calculating the acceleration associated with the movement from the acceleration.

The acceleration A may be calculated by the accelerometer or the like, or may be calculated by an analysis apparatus, a calculation unit of a web server, or the like of a system for managing a health condition of a calf described later.

<Specifying First Resting Period>

In specifying the first resting period, the period in which the acceleration A associated with the movement is the constant M or less is specified. A measurement period of the acceleration for specifying the first resting period is preferably 24 hours or more, more preferably 48 hours or more, still more preferably 72 hours or more, and still more preferably 240 hours or more. In addition, the measurement period of the acceleration may be one year or less, six months or less, three months or less, or one month or less. In a case where there are a plurality of periods in which the acceleration A is the constant M or less within the measurement period of the acceleration, all the periods may be specified as the first resting period, or a part of the periods may be specified as the first resting period.

When the acceleration A is a dimensionless amount obtained by formula (I) above, the constant M is preferably 1, more preferably 0.75, and still more preferably 0.5.

<Specifying Second Resting Period>

In specifying the second resting period, the period having the starting point at the time point after the end point of the first resting period as the start point and in which the acceleration A is the constant M or less is specified. For example, the measurement of the acceleration for specifying the second resting period is started after the end point of the measurement period of the acceleration for specifying the first resting period. Thereafter, the acceleration A in a predetermined time within the measurement period is obtained, and when the value is the constant M or less, the predetermined time may be set as the second resting period. On the other hand, when the value exceeds the constant M, the acceleration A in the next predetermined time is obtained, and when the value is the constant M or less, the predetermined time may be set as the second resting period. Thereafter, the measurement may be stopped or interrupted, or similarly, the measurement of the acceleration, the calculation of the acceleration A, and the specification of the period of the constant M or less may be continued. The predetermined time is preferably 1 minute or more, more preferably 3 minutes or more, and still more preferably 20 minutes or more. This improves a measurement accuracy. On the other hand, the predetermined time is preferably 120 minutes or less, more preferably 90 minutes or less, and still more preferably 60 minutes or less. This makes it possible to quickly determine the body temperature abnormality of the calf.

The specification of each of the resting periods is preferably calculated by the analysis apparatus, a specification unit of a web server, or the like of the system for managing a health condition of a calf described later.

Note that the measurement of the acceleration for specifying the second resting period may be performed continuously with the measurement of the acceleration for specifying the first resting period, or may be performed discontinuously with a time interval.

<Calculating Value Relating to Heartbeat in First Resting Period>

In calculating the value relating to the heartbeat in the first resting period, at least one of the average value of beat intervals, the variation in beat intervals, the beat number, the HF, or the LF/HF in the first resting period is calculated on the basis of the measured beat intervals. It is preferable to calculate at least one of the average value of beat intervals, the variation in beat intervals, or the beat number in the first resting period, it is more preferable to calculate at least one of the average value of beat intervals or the beat number in the first resting period, and it is still more preferable to calculate the average value of beat intervals in the first resting period.

Examples of an average value of beat intervals include an average value of the R-R intervals each of which is the interval between the R wave and the R wave in the electrocardiographic signal, and an average value of the intervals between the pulse wave and the pulse wave. Among them, the average value of the R-R intervals (RRIs) is preferable.

The variation in beat intervals can be obtained by time-analyzing information relating to a time interval such as the RRI. Specifically, the variation in beat intervals is preferably at least one of SDNN, RMSSD, CVRR, NN50, or pNN50. Provided that, the SDNN is a standard deviation of an RRI that is an interval between an R wave and an R wave in an electrocardiographic signal, the RMSSD is a square root of an average value of squares of differences between consecutive adjacent RRIs, the CVRR is a value obtained by dividing a value of the SDNN by an average value of RRIs and multiplying the obtained value by 100, the NN50 is a value indicating a total number of times in which the differences between the consecutive adjacent RRIs exceed 50 me, and the pNN50 is a value indicating a ratio of heartbeats in which the differences between the consecutive adjacent RRIs exceeds 50 ms.

Examples of the beat number in the first resting period include the beat number relating to the heartbeat, the beat number relating to the pulse, and the like. Among them, the beat number is more preferably the beat number of the heartbeat. The beat number relating to the heartbeat can be calculated from the RRI, and can be obtained by, for example, a formula of 60/(RRI×0.001).

The HF and the LF/HF are usually used as indices of parasympathetic nerve activity and sympathetic nerve activity, but can be used as indices of the body temperature abnormality. The HF and the LF can be each obtained, for example, by calculating a power spectrum of the RRI, the spectrum obtained by including performing frequency analysis, and integrating a power in a predetermined frequency region. For example, the LF can be obtained by definite integration from the frequencies Lf1 to Lf2, and the HF can be obtained by definite integration of the power spectrum from the frequencies Hf1 to Hf2. Here, Hf1>Lf1 and Hf2>Lf2 hold.

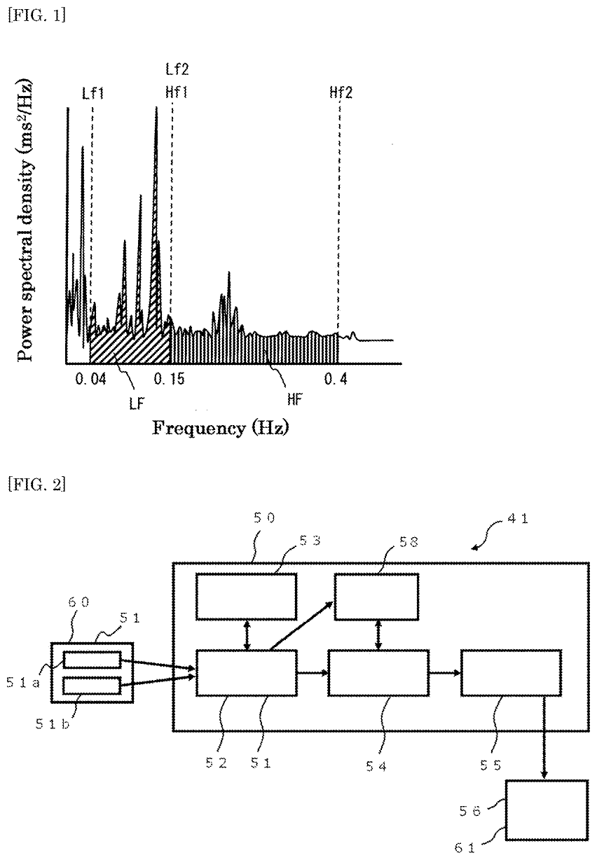

A detailed method for calculating the LF and the HF will be described with reference to FIG. 1 which is an explanatory diagram of power spectrum integration. In FIG. 1, a vertical axis represents a power spectral density (unit: ms²/Hz), and a horizontal axis represents a frequency (unit: Hz). The LF is a value obtained by performing definite integration of a power spectrum F², for example, from 0.04 Hz (Lf1) to 0.15 Hz (Lf2), and is an area of a portion hatched by oblique lines in FIG. 1. On the other hand, the HF is a value obtained by performing definite integration of the power spectrum F², for example, from 0.15 Hz (Hf1) to 0.40 Hz (Hf2), and is an area of a portion hatched by vertical lines in FIG. 1. In FIG. 1, an integration range is set such that both Lf2 and Hf1 are equal to 0.15 Hz, but is not limited to the range. Relationships of Lf1<Hf1 and Lf2<Hf2 are preferably satisfied, and a relationship of Hf1>Lf2 is more preferably satisfied. In addition, the integration range of the LF may be 0.0133 to 0.30 Hz and the integration range of the HF may be 0.30 to 0.80 Hz.

It is preferable that the integration range of the LF includes at least 0.10 Hz and Lf1<0.10<Lf2 holds. Further, Ifl is more preferably 0.01 to 0.05 Hz. Lf2 is more preferably 0.10 to 0.34 Hz. It is preferable that the integration range of the HIF includes at least 0.35 Hz and Hf1<0.35<Hf2 holds. Hf1 is more preferably 0.10 to 0.34 Hz. Hf2 is more preferably 0.38 to 0.90 Hz. In calculating the LF and the HF described above, for example, WO-A-2016-031650 and the like can be referred to.

The value relating to the heartbeat in the first resting period is preferably calculated by the analysis apparatus or the calculation unit of the web server in the system for managing a health condition of a calf described later.

<Calculating Value Relating to Heartbeat in Second Resting Period>

The value relating to the heartbeat in the second resting period can be calculated in a similar manner to the calculation of the value relating to the heartbeat in the first resting period. The value relating to the heartbeat in the second resting period is preferably calculated by the analysis apparatus or the calculation unit of the web server in the system for managing a health condition of a calf described later.

The beat number in the first resting period and the beat number in the second resting period are preferably the heart rate or the pulse rate, and more preferably the heart rate. This makes it easier to determine the body temperature abnormality.

<Issuing Warning for Notifying Abnormality of Body Temperature>

In issuing the warning, when the magnitude relationship of at least one of formulae (1) to (5) above is satisfied, the warning for notifying abnormality of the body temperature of the calf is issued. It is preferable to issue a warning when the right side becomes 0.95 times or less the left side in formulae (1), (3), or (4) above, and it is more preferable to issue a warning when the right side becomes 0.93 times or less the left side. In addition, it is preferable to issue the warning when a right side is 1.05 times or more a left side in formulae (2) or (5) above, and it is more preferable to issue the warning when the left side is 1.07 times or more the right side.

In issuing the warning, it is preferable to issue the warning when the magnitude relationship of at least one of formulae (1) to (3) above is satisfied, it is more preferable to issue the warning when the magnitude relationship of at least one of formulae (1) or (2) above is satisfied, and it is still more preferable to issue the warning when the magnitude relationship of formula (1) above is satisfied.

The warning may be perceptible information or a warning signal for the apparatus to perform a necessary treatment. Examples of the perceptible information include sound, vibration, and image. A warning apparatus may be used for the warning, and examples of the warning apparatus include a sound generator, a vibration generator, and a display. In issuing the warning, it is preferable that the warning is issued from a warning unit of the system for managing a health condition of a calf described later.

<Raising or Lowering Body Temperature>

In raising or lowering the body temperature, the body temperature of the calf is raised or lowered after the warning is issued. Since the body temperature of the calf easily decreases, it is preferable to raise the body temperature of the calf in this step.

It is more preferable to raising the body temperature of the calf by using a heat retaining garment, heating equipment, or a hot-water supply machine. Examples of the heat retaining garment include a jacket for a calf. Examples of the heating equipment include a heater such as a fan heater, a far infrared heater, or an oil heater, and an air conditioner. In the case of using a hot-water supply machine, examples thereof include a mode in which the calf is warmed in a bath to be immersed in hot water, and a treatment in which hot water is supplied to a hot water bottle, and the calf is warmed by the hot water bottle.

Furthermore, treatments such as reducing an amount of feed such as milk to be given to the calf, weaning, oral feeding of an oral electrolyte, feeding of feed, administration of a probiotic agent, and administration of an antidiarrheal may be performed. Two or more of these treatments may be used in combination.

On the other hand, when heat stroke or the like is suspected, the body temperature of the calf may be lowered after generation of the warning. When such a treatment is performed, an electric fan, an air conditioner, a water supply machine, or the like can be used.

These treatments may be performed by the owner or the like who has received the warning, the apparatus that has received the warning signal may perform the treatment directly, or the apparatus that has received the warning signal may perform the treatment via another apparatus. For example, the above treatment may be performed by a thermoregulation unit of the system for managing a health condition of a calf described later.

The calf is preferably cattle having an age less than 9 months. Since the calf has a thin subcutaneous fat and a small body, heat is easily deprived from a body surface, and since a rumen is not yet developed, fermentation heat by microorganisms cannot be obtained, the calf is easily subjected to cold stress, and an immune function easily deteriorates. In addition, the calf has a high risk of infection due to insufficient nutrition necessary for growth. On the other hand, the calf is prone to heat stroke in places having poor air permeability, places easily exposed to sunlight, and the like. Therefore, the calf to be managed for the health condition is preferably less than 9 months of age, and more preferably 6 months or less of age.

It is preferable to measure the beat intervals using a biological information measuring garment. The garment preferably has a beat intervals measuring instrument that measures the beat intervals such as an electrocardiograph. A form of the garment is not particularly limited, and a belt-shaped article such as underwear or a belt is preferable. In addition, the garment preferably covers at least one of the chest, the abdomen, a leg, a neck, and a head, more preferably covers the chest and/or the abdomen, and still more preferably covers the chest. In addition, the garment preferably includes a detachable electronic unit.

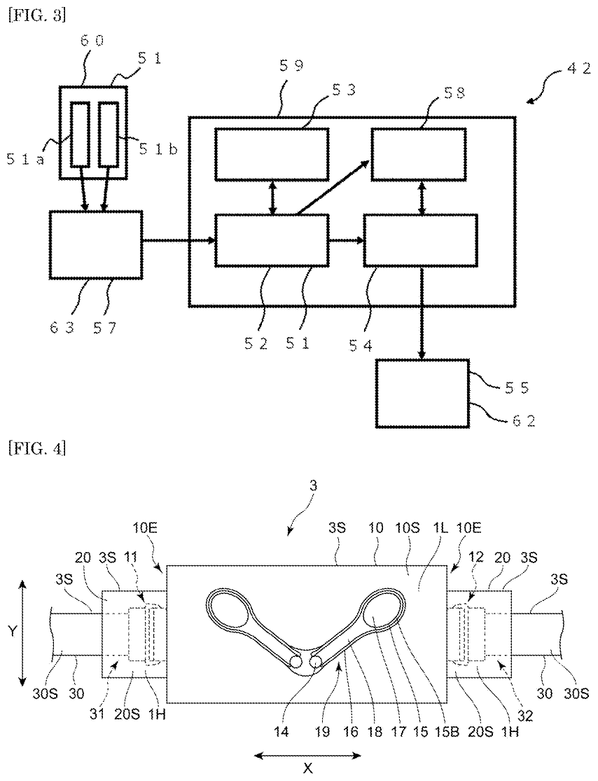

Examples of the biological information measuring garment include a belt-like biological information measuring garment 3 illustrated in FIG. 4. The belt-like biological information measuring garment 3 has first clothing fabric 10 and electrodes 15 provided on a skin-side surface 10S of the first clothing fabric 10. The belt-like biological information measuring garment 3 preferably further has a belt-like member 30 provided at end portions 10E of the first clothing fabric 10 in a body circumferential direction X. The first clothing fabric 10 preferably has a first coupling member 11 and a second coupling member 12 provided at the end portions 10E in the body circumferential direction X. Examples of these members include hook-and-loop fasteners such as Magic Tape (registered trademark) and Free Magic Tape (registered trademark), buckles, hooks, loops, and the like.

The belt-like member 30 preferably has a third coupling member 31 and a fourth coupling member 32 at end portions in the body circumferential direction X. Examples of these members include hook-and-loop fasteners such as a female buckle, a male buckle, Magic Tape (registered trademark), and Free Magic Tape (registered trademark), hooks, loops, and the like.

In FIG. 4, the third coupling member 31 and the fourth coupling member 32 that are male buckles are coupled to be incorporated in female buckles of the first coupling member 11 and the second coupling member 12 provided at the end portions 10E in the body circumferential direction X of the belt-like biological information measuring garment 3 for a calf, respectively.

Preferably, the belt-like biological information measuring garment 3 preferably further has second clothing fabric 20 provided at the end portions 10E of the first clothing fabric 10 in the body circumferential direction X and/or the belt-like member 30 such that the second clothing fabric 20 is located on a skin side of the belt-like member 30. In FIG. 4, one end portion of the second clothing fabric 20 in the body circumferential direction X is sewn and fixed to each of the end portions 10E, and has a flap shape. Further, the second clothing fabric 20 may be fixed to the belt-like member 30.

The second clothing fabric 20 may be movably provided on the first clothing fabric 10, or may be movably provided on the belt-like member 30. Examples of an aspect of the movable second clothing fabric 20 include an aspect in which the second clothing fabric 20 has a loop shape, and the belt-like member 30 passes through the loop.

A skin-side surface 3S of the belt-like biological information measuring garment 3 preferably has high friction portions 1H having an average friction coefficient MIU of 0.40 or more. As a result, the belt-like biological information measuring garment 3 is hardly displaced. In FIG. 4, while skin-side surfaces 20S of the second clothing fabric 20 has the high friction portions 1H, each of the skin-side surfaces of the first clothing fabric 10 may have the high friction portion 1H, and a skin-side surface 30S of the belt-like member 30 may have the high friction portion 1H.

The high friction portions 1H are preferably each a portion where fine fibers are exposed. Preferred examples of the fine fibers include microfibers and nanofibers. A single fiber diameter of the fine fibers is preferably 8 μm or less, more preferably 1000 nm or less, still more preferably less than 1000 nm, still more preferably 800 nm or less, and particularly preferably 750 nm or less. On the other hand, the single fiber diameter may be 100 nm or more, 300 nm or more, or 500 nm or more.

The skin-side surface 3S of the belt-like biological information measuring garment 3 preferably further has a low friction portion 1L having an average friction coefficient MIU less than 0.40. This can make it easy to adjust a position of the belt-like biological information measuring garment 3. In FIG. 4, while the skin-side surfaces 10S of the first clothing fabric 10 has the low friction portion 1L, each of the skin-side surfaces of the second clothing fabric 20 may have the low friction portion 1L, and the skin-side surface 30S of the belt-like member 30 may have the low friction portion 1L.

The belt-like biological information measuring garment 3 preferably has a tensile strength of 2.8 N/cm or less at the time of 5% elongation and a tensile strength of 4.0 N/cm or less at the time of 10% elongation. When the tensile strength is in the above range, the belt can be elongated according to growth of the calf. The tensile strength can be measured, for example, by the following procedure. First, the belt-like biological information measuring garment 3 is nipped with chucks such that a center of the first clothing fabric 10 is located at a center between the chucks of a tensile tester and a distance between the chucks is 45 cm, then the belt-like biological information measuring garment 3 is elongated in the body circumferential direction X under conditions of a load cell of 1 kN and a tensile speed of 100 mm/min, and then loads (N) at the time of 2.25 cm elongation (at the time of 5% elongation) and at the time of 4.5 cm elongation (at the time of 10% elongation) are obtained. Further, the tensile strength at the time of 5% elongation (N/cm) and the tensile strength at the time of 10% elongation (N/cm) can be obtained by dividing the loads (N) by an average width of the belt-like biological information measuring garment 3 between the chucks.

A number of the electrodes 15 is not limited to one, and two or more electrodes may be provided on the skin-side surface 10S of the first clothing fabric 10. Each of the electrodes 15 preferably includes a first insulating layer 16 formed on the skin-side surface 10S of the first clothing fabric 10 and a conductive layer 17 formed on the first insulating layer 16.

A portion other than the electrodes 15 in the conductive layer 17 or a portion other than electronic equipment connection portions 14 and the electrodes 15 in the conductive layer 17 is preferably covered with a second insulating layer 18.

Wiring 19 is preferably provided on the skin-side surface 10S of the first clothing fabric 10. The wiring 19 preferably includes the first insulating layer 16, the conductive layer 17 formed on the first insulating layer 16, and the second insulating layer 18 formed on the conductive layer 17. Biological information acquired by the electrodes 15 can be transmitted via the wiring 19.

Another layer such as an adhesive layer may be present between the skin-side surface 10S and the first insulating layer 16. In addition, another layer such as a hot melt layer may be present between the first insulating layer 16 and the conductive layer 17. Another layer such as a hot melt layer may be present between the conductive layer 17 and the second insulating layer 18.

The electronic equipment connection portions 14 are formed of the first insulating layer 16 provided on the skin-side surface 10S of the first clothing fabric 10 and the conductive layer 17 provided on the first insulating layer 16. In each of the electronic equipment connection portions 14, electronic equipment can be attached to a front surface on the opposite side of the skin-side surface 10S of the first clothing fabric 10 via a connector such as, for example, a snap fastener. In addition, a protective layer may be formed so as to cover a skin-side surface of the electronic equipment connection portion 14.

Details of the electrode 15, the first insulating layer 16, the conductive layer 17, the second insulating layer 18, the wiring 19, and the like can be referred to JP-A-2020-100903.

These methods for managing a health condition of a calf can be implemented using, for example, the system for managing a health condition of a calf or an apparatus for managing a health condition of a calf, which will be described below. At least some of the steps of the method for managing a health condition of a calf may be executed by the owner or the like of the calf, and all the steps may be executed by the system or the apparatus for managing a health condition of a calf.

A system for managing a health condition of a calf according to an embodiment of the present invention includes: a measurement unit that measures beat intervals and acceleration associated with movement; a specification unit that specifies a first resting period in which the acceleration is a constant M or less, and specifies a second resting period that is a period having a starting point at a time point after an end point of the first resting period and in which the acceleration is the constant M or less; a calculation unit that calculates at least one of an average value of beat intervals, a variation in beat intervals, a beat number, HF, or LF/HF in the first resting period, and calculates at least one of an average value of beat intervals, a variation in beat intervals, a beat number, HF, or LF/HF in the second resting period; a determination unit that generates a warning signal when a magnitude relationship of at least one of formulae (1) to (5) below is satisfied; and a warning unit that issues a warning for notifying abnormality of a body temperature of the calf from the warning signal:

the average value of beat intervals in the first resting period>the average value of beat intervals in the second resting period (1)

the beat number in the first resting period<the beat number in the second resting period (2)

the variation in beat intervals in the first resting period>the variation in beat intervals in the second resting period (3)

the HF in the first resting period>the HF in the second resting period (4)

the LF/HF in the first resting period<the LF/HF in the second resting period (5)
provided that, each LF is a value obtained by performing definite integration of a power spectrum from frequencies L_{f1} to L_{f2}, the power spectrum being obtained by including performing frequency spectrum conversion on beat intervals, and the HF is a value obtained by performing definite integration of the power spectrum from frequencies H_{f1} to H_{f2}, where H_{f1}>L_{f1} and H_{f2}>L_{f2} are satisfied.

As described above, the system for managing a health condition of a calf calculates at least one of the average value of beat intervals, the variation in beat intervals, the beat number, the HF, or the LF/HF (hereinafter, it may be simply referred to as value relating to the heartbeat) in each of the resting periods, and issues a warning to notify the body temperature abnormality of the calf when at least one of the magnitude relationships of formulae (1) to (5) above is satisfied. This makes it possible to determine the occurrence of the abnormality of the body temperature of the calf.

For the beat intervals, the acceleration associated with the movement (acceleration A), the specification of the resting periods, the value relating to the heartbeat, formulae (1) to (5), and the like, the description of the method for managing a health condition of a calf can be referred to.

Hereinafter, a configuration and a function of the system for managing a health condition of a calf 41 according to a first embodiment will be described with reference to FIG. 2. Description of the matters described in the method for managing a health condition of a calf will be omitted.

As illustrated in FIG. 2, the system for managing a health condition of a calf 41 has a measurement instrument 60 and an analysis apparatus 50. As illustrated in FIG. 2, the system for managing a health condition of a calf 41 may further include a thermoregulator 61. In addition, the analysis apparatus 50 has a measurement unit 51, a calculation unit 52, a specification unit 53, a determination unit 54, and a warning unit 55, and may have a storage unit 58. Further, the measurement instrument 60 may have the beat interval measuring unit 51a and the acceleration measuring unit 51b.

The measurement instrument 60 is attached to the calf, and a beat interval measuring unit 51a and an acceleration measuring unit 51b of the measurement unit 51 measure the beat intervals and the acceleration. Subsequently, measurement results are transmitted to the calculation unit 52, and the calculation unit 52 calculates the acceleration A associated with the movement of the calf. In this case, the calculation unit 52 functions as a measurement unit for measuring the acceleration A. On the other hand, the acceleration A may be measured by the measurement unit 51. Subsequently, the specification unit 53 specifies the first resting period in which the acceleration A is the constant M or less. Furthermore, the calculation unit 52 calculates the value relating to the heartbeat in the first resting period. The calculated value is stored in the storage unit 58 as a value relating to the first resting period.

Similarly, in the period having the starting point at the time point after the end point of the first resting period, the beat interval measuring unit 51a and the acceleration measuring unit 51b measure the beat intervals and the acceleration. Subsequently, measurement results are transmitted to the calculation unit 52, and the calculation unit 52 calculates the acceleration A associated with the movement of the calf. In this case, the calculation unit 52 functions as a measurement unit for measuring the acceleration A. On the other hand, the acceleration A may be measured by the measurement unit 51. Subsequently, the specification unit 53 specifies the second resting period in which the acceleration A associated with the movement is the constant M or less. Furthermore, the calculation unit 52 calculates the value relating to the heartbeat in the second resting period. The calculated value is stored in the storage unit 58 as a value relating to the second resting period.

Next, the determination unit 54 compares the value relating to the first resting period and the value relating to the second resting period stored in the storage unit 58, and in the case where the magnitude relationship of at least one of formulae (1) to (5) is satisfied, the determination unit 54 generates the warning signal. Next, the warning unit 55 that has received the warning signal issues the warning for notifying abnormality of the body temperature of the calf.

Examples of the measurement instrument 60 include an electrocardiograph that measures the heartbeat of the calf, a heartbeat sensor, a pulse wave sensor that measures a pulse, an acceleration sensor, and the like. These can be used singly or in combination of two or more kinds thereof. The measurement unit 51 may measure the beat intervals such as the RRIs, and the acceleration. The beat interval measuring unit 51a and the acceleration measuring unit 51b do not need to be configured by the same measurement instrument, and may be configured by different measurement instruments. The measurement instrument 60 may be formed integrally with the analysis apparatus 50.

As illustrated in FIG. 2, the calculation unit 52, the specification unit 53, the determination unit 54, and the storage unit 58 may be provided as configuration units of the analysis apparatus 50, and at least a part of them may be provided as configuration units of the measurement instrument 60. The units may be configured of a processor such as a central processing unit (CPU) or a microprocessor (MPU), a storage medium such as a random access memory (RAM) or a read only memory (ROM), a logic circuit such as a field programmable gate array (FPGA), a large-scale integration (LSI), or an application specific integrated circuit (ASIC), a part or all of them, and the like. In addition, the analysis apparatus 50 may have an auxiliary storage device such as a hard disk drive (HDD) or a flash memory, and a portable recording medium such as a CD-ROM, a DVD disk, or a USB memory.

The warning issued from the warning unit 55 may be perceptible information, or may be a signal for a thermoregulation unit 56 to perform a treatment for raising or lowering the body temperature of the calf. Examples of the perceptible information include sound, vibration, and image. Examples of a warning apparatus configuring the warning unit 55 include a sound generator, a vibration generator, and a display. These may be used singly or in combination of two or more kinds thereof. In addition, the warning apparatus may be provided inside the analysis apparatus 50 or may be provided outside the analysis apparatus 50.

The transmission of the signal from the measurement unit 51 to the calculation unit 52 may be wired or wireless. In addition, the analysis apparatus 50 may have a reception unit (not illustrated) that receives the signal from the measurement unit 51. Furthermore, the analysis apparatus 50 may have a storage unit (not illustrated) that stores the measurement result between the reception unit and the calculation unit 52. Furthermore, the calculation unit 52 may appropriately extract data from the storage unit and perform noise cut processing.

Examples of the analysis apparatus 50 include electronic equipment such as, for example, a smartphone, a tablet terminal, a smart watch, and a computer.

The thermoregulation unit 56 of the thermoregulator 61 receives a signal from the warning unit 55 in a wired or wireless manner, and performs the treatment to raise or lower the body temperature of the calf. Note that the owner or the like of the calf who has received the warning from the warning unit 55 may operate the thermoregulation unit 56. In addition, the owner or the like may directly perform a treatment such as putting the heat retaining garment on the calf, or supplying water. The thermoregulator 61 may be a heater, an air conditioner, a water supply machine, a water sprinkler, an electric fan, or the like that can be operated by the signal from the warning unit 55. Preferably, the signal is wirelessly transmitted from the warning unit 55 to the thermoregulation unit 56.

The system for managing a health condition of a calf 41 may have a respiration rate measuring instrument as the measurement instrument 60. Symptoms of cough can also be detected by changes in respiration. Specifically, as means for measuring a respiration rate, the respiration rate can be measured by attaching a displacement sensor that detects a change in length to a site where a circumferential length changes due to respiration in the calf. Examples of the displacement sensor include displacement sensors of a type in which a resistance is changed by displacement and a type in which a capacitance is changed.

Next, a system for managing a health condition of a calf 42 according to a second embodiment will be described with reference to FIG. 3. Components similar to those of the system for managing a health condition of a calf 41 according to the first embodiment are denoted by similar reference signs, and description thereof will be omitted.

As illustrated in FIG. 3, the system for managing a health condition of a calf 42 has the measurement instrument 60, a web server 59, and a warning apparatus 62, and may have a transceiver 63. In addition, the web server 59 has the measurement unit 51, the calculation unit 52, the specification unit 53, and the determination unit 54, and may include the storage unit 58. The warning apparatus 62 has the warning unit 55.

The measurement instrument 60 of the system for managing a health condition of a calf 42 is attached to the calf, and the beat interval measuring unit 51a and the acceleration measuring unit 51b of the measurement unit 51 measure the beat intervals and the acceleration. Subsequently, the measurement results are transmitted to a reception unit of a transmission and reception unit 57, and is transmitted from a transmission unit of the transmission and reception unit 57 to the web server 59. The calculation unit 52 of the web server 59 calculates the acceleration A associated with the movement. In this case, the calculation unit 52 functions as a measurement unit for measuring the acceleration A. On the other hand, the acceleration A may be measured by the measurement unit 51. Subsequently, the specification unit 53 of the web server 59 specifies the first resting period in which the acceleration A is the constant M or less. Furthermore, the calculation unit 52 calculates the value relating to the heartbeat in the first resting period. The calculated value is stored in the storage unit 58 as a value relating to the first resting period.

Similarly, in the period having the starting point at the time point after the end point of the first resting period, the beat interval measuring unit 51a and the acceleration measuring unit 51b of the measurement unit 51 measure the beat intervals and the acceleration. Subsequently, the measurement results are sent to the reception unit of the transmission and reception unit 57, and is transmitted from the transmission unit of the transmission and reception unit 57 to the web server 59. The measurement results are transmitted to the calculation unit 52 of the web server 59, and the calculation unit 52 calculates the acceleration A. In this case, the calculation unit 52 functions as a measurement unit for measuring the acceleration A. On the other hand, the acceleration A may be measured by the measurement unit 51. Subsequently, the specification unit 53 specifies the second resting period in which the acceleration A is the constant M or less. Furthermore, the calculation unit 52 calculates the value relating to the heartbeat in the second resting period. The calculated value is stored in the storage unit 58 as the value relating to the second resting period. These pieces of data are sent to the determination unit 54 of the web server 59, and the determination unit 54 generates the warning signal when the magnitude relationship of at least one of formulae (1) to (5) above is satisfied. Next, the warning unit 55 of the warning apparatus 62 that has received the warning signal from the web server 59 issues the warning for notifying the abnormality of the body temperature of the calf.

Examples of the transceiver 63 configuring a transition and reception unit 57 include a transceiver of a short-range wireless communication technology Bluetooth or the like. As a result, the transmission from the measurement unit 51 to the calculation unit 52 can be performed wirelessly. Note that the transmission may be performed by wire.

Examples of the web server 59 include a rental server and a cloud server, and the cloud server is preferable.

The transmission from the determination unit 54 to the warning unit 55 can be performed wirelessly. Note that the transmission may be performed by wire.

The web server 59 may have a reception unit (not illustrated) that receives the signal from the measurement unit 51, and may have, between the reception unit and the calculation unit 52, a storage unit (not illustrated) that stores the measurement result. Furthermore, the calculation unit 52 may appropriately extract data from the storage unit and perform noise cut processing.

Examples of the warning apparatus 62 configuring the warning unit 55 include electronic equipment such as a smartphone, a tablet terminal, a smart watch, and a computer. Among them, the smartphone, the tablet terminal, and the smartwatch are preferable.

A garment according to an embodiment of the present invention includes at least a part of the system for managing a health condition of a calf. The garment for managing a health condition of a calf preferably has the measurement unit 51. The garment for managing a health condition of a calf may further have the calculation unit 52, the specification unit 53, and the determination unit 54, may further have the warning unit 55, and may further have the thermoregulation unit 56. A form of the garment for managing a health condition of a calf is not particularly limited, and a belt-like article such as underwear or a belt is preferable. In addition, the garment preferably covers at least one site of the chest, the abdomen, the leg, the neck, and the head, more preferably covers the chest and/or the abdomen. In addition, the garment preferably includes a detachable electronic unit.

An apparatus for managing a health condition of a calf according to an embodiment of the present invention, includes: a measurement unit that measures beat intervals and acceleration associated with movement; a specification unit that specifies a first resting period in which the acceleration is a constant M or less, and specifies a second resting period that is a period having a starting point at a time point after an end point of the first resting period and in which the acceleration is the constant M or less; a calculation unit that calculates at least one of an average value of beat intervals, a variation in beat intervals, a beat number, H F, or LF/HF in the first resting period, and calculates at least one of an average value of beat intervals, a variation in beat intervals, a beat number, HF, or LF/HF in the second resting period; and a determination unit that generates a warning signal when a magnitude relationship of at least one of formulae (1) to (5) below is satisfied:

the average value of beat intervals in the first resting period>the average value of beat intervals in the second resting period (1)

the beat number in the first resting period<the beat number in the second resting period (2)

the variation in beat intervals in the first resting period>the variation in beat intervals in the second resting period (3)

the HF in the first resting period>the HF in the second resting period (4)

the LF/HF in the first resting period<the LF/HF in the second resting period (5)
provided that, each LF is a value obtained by performing definite integration of a power spectrum from frequencies L_{f1} to L_{f2}, the power spectrum being obtained by including performing frequency spectrum conversion on beat intervals, and the HF is a value obtained by performing definite integration of the power spectrum from frequencies H_{f1} to H_{f2}, where H_{f1}>L_{f1} and H_{f2}>L_{f2} are satisfied.

The apparatus for managing a health condition of a calf according to the embodiment of the present invention has the measurement unit, the calculation unit, the specification unit, the determination unit, and the warning unit. For details of the units, the beat intervals, the acceleration, the specification of the resting periods, the value relating to the heartbeat, formulae (1) to (5), and the like, the description of the method for managing a health condition of a calf, the system for managing a health condition of a calf 41 according to the first embodiment, and the like can be referred to.

Specific examples of the apparatus for managing a health condition of a calf include an apparatus having the measurement unit 51, the calculation unit 52, the specification unit 53, and the determination unit 54, and the warning unit 55 of the system for managing a health condition of a calf 41. Furthermore, the apparatus for managing a health condition of a calf may have the thermoregulation unit 56 and the transmission and reception unit 57. In addition, the apparatus for managing a health condition of a calf may be an apparatus including the measurement instrument 60 and the analysis apparatus 50.

The present application claims the benefit of priority based on JP-A-2020-193645 filed on Nov. 20, 2020. The entire contents of the specification of JP-A-2020-193645 filed on Nov. 20, 2020 are incorporated herein by reference.

### EXAMPLES

Hereinafter, the present invention will be more specifically described with reference to examples, but the present invention is not limited by examples below, and can be carried out with modifications within a range that can conform to the gist described above and below, and all of them are included in the technical scope of the present invention.

### Example 1

Clothing fabric having a shape similar to that of the first clothing fabric 10 illustrated in FIG. 4 was produced. First, bonding double face cloth (W220/2028A) having a jersey surface and a mesh surface and manufactured by MASUMITETU Inc. was cut into a rectangular shape of 28 cm×10 cm. Subsequently, electrodes were provided on the jersey surface (skin-side surface). Details are as described below.

As a resin, 20 parts by mass of nitrile rubber (Nipol DN003 manufactured by Zeon Corporation) was dissolved in 80 parts by mass of isophorone to prepare an NBR solution. Tb 100 parts by mass of this NBR solution, 110 parts of silver powder (“Agglomerated Silver Powder G-35” manufactured by Dowa Electronics Materials Co., Ltd., average powder diameter 5.9 μm) were mixed and kneaded with a triple roll mill to obtain a stretchable silver paste.

Next, the stretchable silver paste was applied onto a release sheet and dried in a hot air drying oven at 120° C. for 30 minutes or more to prepare a sheet-shaped conductive layer with the release sheet. The conductive layer of this sheet-shaped conductive layer with the release sheet corresponds to the above-described conductive layer 17.

Next, a polyurethane hot-melt sheet was bonded to a surface of the conductive layer of the sheet-shaped conductive layer with the release sheet, and then the release film was peeled off to obtain a sheet-shaped conductive layer with a polyurethane hot-melt sheet. The polyurethane hot-melt sheet was laminated using a hot press machine under a condition of a pressure of 0.5 kg/cm², a temperature of 130° C., and a pressing time of 20 seconds.

Next, a polyurethane hot-melt sheet side of the sheet-shaped conductive layer with a polyurethane hot-melt sheet having a length of 12 cm was laminated on a polyurethane hot-melt sheet having a length of 13 cm to produce a laminate of the polyurethane hot-melt sheet and the sheet-shaped conductive layer. The polyurethane hot-melt sheet corresponds to the above-described first insulating layer 16.

Next, the second insulating layer 18 was formed by covering the first insulating layer 16 and the conductive layer 17 using the same polyurethane hot-melt sheet as that used to form the first insulating layer 16 such that a part of a skin-side surface of the conductive layer 17 was exposed in an elliptical shape (major axis: 4 cm, minor axis: 3 cm) at one end portion in a length direction of the conductive layer 17 and a part of the skin-side surface of the conductive layer 17 was exposed in a circular shape (diameter: 1 cm) at another end portion. At this time, the first insulating layer 16 and the conductive layer 17 were covered such that an outer edge of the second insulating layer 18 was located outside outer edges of the first insulating layer 16 and the conductive layer 17. As a result, an electrode wiring sheet including the electronic equipment connection portion 14 having a laminated structure of the first insulating layer 16/the conductive layer 17 and exposing the conductive layer 17, the wiring 19 having a laminated structure of the first insulating layer 16/the conductive layer 17/the second insulating layer 18, and the electrode 15 having the laminated structure of the first insulating layer 16/the conductive layer 17, and exposing the conductive layer 17 was produced.

Next, the two electrode wiring sheets were pasted at predetermined positions on the jersey surface (skin-side surface) of the first clothing fabric 10 so as to be bilaterally symmetrical as illustrated in FIG. 4. Further, at the electronic equipment connection portions 14, the electrocardiogram and an acceleration measuring apparatus was connected to a front side via snap hooks as connectors to obtain the first clothing fabric 10 with the electrode wiring.

Subsequently, grosgrain 7000 made of polyester, having a width of 5 cm and a length of 6 cm and manufactured by INOUE RIBBON INDUSTRY Co., Ltd. was passed through a through hole of a female buckle into a loop shape, and both end portions in a length direction were sewn to the one end portion 10E of the first clothing fabric 10 with the electrode wiring to form the first coupling member 11 as illustrated in FIG. 4. Further, the second coupling member 12 was formed at the other end portion 10E in a similar manner. Furthermore, an end portion in a length direction of a high grip tape 1760 having a width of 6.3 cm and a length of 10 cm, manufactured by INOUE RIBBON INDUSTRIAL Co., Ltd. containing polyester fibers and polyurethane fibers was fixed to the one end portion 10E of the first clothing fabric 10 in the body circumferential direction X by sewing so as to be located on the skin side with respect to the first coupling member 11 and the second coupling member 12, thereby forming a flap shape to produce the second clothing fabric 20. Furthermore, the second clothing fabric 20 was formed at the other end portion 10E in a similar manner. Furthermore, the outer edges of the first clothing fabric 10 and the second clothing fabric 20 were piped using a binder tape W1250 manufactured by ASAKURA SENPU Co., Ltd. Furthermore, male buckles were attached to both end portions of a plane elastic 1100 containing polyester and polyurethane, having a width of 5 cm and a length of 90 cm, and manufactured by INOUE RIBBON INDUSTRY Co., Ltd. to obtain the belt-like member 30 including the third coupling member 31 and the fourth coupling member 32. The third coupling member 31 and the fourth coupling member 32 of the belt-like member 30 were coupled to the first coupling member 11 and the second coupling member 12 of the first clothing fabric 10, respectively, to obtain the belt-like biological information measuring garment 3.

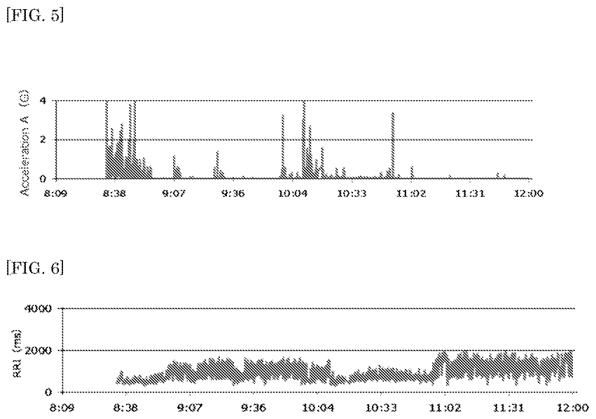

The belt-like biological information measuring garment 3 was worn on the chest of a Japanese black calf (castrated male, chest circumference: 133 cm, age: 6 months). Furthermore, the electrocardiogram and the acceleration measuring apparatus of the belt-like biological information measuring garment 3 was used as the measurement instrument 60 as illustrated in FIG. 2, and the measurement instrument 60, the transceiver 63, the web server 59, and the warning apparatus 62 were wirelessly connected. As a result, the system for managing a health condition of a calf 42 according to the second embodiment was configured, and the beat intervals and the acceleration were measured for 3 days. Data obtained by the measurement instrument 60 was transmitted to the web server 59 via the transceiver 63, and the web server 59 calculated the acceleration A of formula (I) above on the basis of a predetermined program, specified a period in which the acceleration A was 0.5 or less as the first resting period, and obtained an average value of the RRIs in the first resting period. As a result, the average value of the RRIs was 1270 ms. Similarly, the beat intervals and the acceleration were measured in the period after the end point of the first resting period, the acceleration A of formula (I) was calculated, a period in which the acceleration A was 0.5 or less was specified, and 3 minutes of the period was set as the second resting period to obtain an average value of the RRIs. As a result, the average value of the RRIs was 1306 ms. The measurement was continued similarly even after the lapse of 3 minutes. The measurement was performed from 8:30 to 12:00. FIG. 5 is a graph illustrating a relationship between the acceleration A and a measurement time for specifying the second resting period, and FIG. 6 is a graph illustrating a relationship between the RRI and the measurement time for specifying the second resting period. During the measurement period from 8:30 to 12:00, the average value of the RRIs per 3 minutes did not fall below a reference value of 1270 ms, and the magnitude relationship of formula (1) above was not satisfied. Therefore, an alarm was not notified from the warning apparatus 62. In addition, the calf had no symptoms such as disordered breathing and shaking from 8:30 to 12:00.

### Example 2

In a similar manner as in example 1, the belt-like biological information measuring garment 3 was worn on the chest of a Japanese black calf (female, chest circumference: 89 cm, age: 1 month), and the beat intervals and acceleration were measured for 10 days. Obtained data was transmitted from the measurement instrument 60 to the web server 59 via the transceiver 63, and the web server 59 calculated the acceleration A of formula (I) above on the basis of the predetermined program, specified a period in which the acceleration A was 0.5 or less as the first resting period, and obtained an average value of the RRIs in the first resting period. As a result, the average value of the RRIs was 486 ms. Similarly, the beat intervals and the acceleration were measured in a period of 13:00 to 15:00 after the end point of the first resting period, the acceleration A of formula (I) was calculated, a period in which the acceleration A was 0.5 or less was specified, and 30 minutes of the period was set as the second resting period to obtain the average value of the RRIs. As a result, the average value of the RRIs was 450 ms. Since the average value of the RRIs per 30 minutes fell below a reference value of 486 ms and the magnitude relationship of formula (1) above was satisfied, an alarm was notified from the warning apparatus 62. Since symptoms such as disordered breathing and shaking were observed in the calf, the heat-retaining garment was quickly put on the calf. Furthermore, a high-calorie feed was fed. As a result, the symptoms of the calf subsided, and the symptoms did not deteriorate to the extent that administration of an antibiotic agent was necessary.

### Reference Example 1

The system for managing a health condition of a calf 42 according to the second embodiment was configured in a similar manner to in example 1 except that a seal type electrocardiogram measuring electrodes were pasted to a chest of a dog (male, golden retriever, age: 8 months) instead of the electrode wiring sheet, and the beat intervals and the acceleration were measured for 5 days. Data obtained by the measurement instrument 60 was transmitted to the web server 59 via the transceiver 63, and the web server 59 calculated the acceleration A of formula (I) above on the basis of the predetermined program, specified a period in which the acceleration A was 0.5 or less as the first resting period, and obtained the average value of the RRIs in the first resting period. As a result, the average value of the RRIs was 836 ms. Similarly, the beat intervals and the acceleration were measured in a period of 21:00 to 6:00 after the end point of the first resting period, the acceleration A of formula (I) was calculated, a period in which the acceleration A was 0.5 or less was specified, and 30 minutes of the period was set as the second resting period to obtain the average value of the RRIs. As a result, the average value of the RRIs was 770 ms. Since the average value of the RRIs per 30 minutes fell below a reference value of 836 ms and the magnitude relationship of formula (1) above was satisfied, an alarm was notified from the warning apparatus 62. Symptoms such as disordered breathing and shaking were observed in the dog, and heat stroke was suspected from the measurement in a summer season. Therefore, the whole body was quickly cooled by pouring cold water. As a result, the symptoms of the dog subsided, and the symptoms did not deteriorate to the extent that drip infusion was necessary.

### DESCRIPTION OF REFERENCE SIGNS

3 belt-like biological Information measuring garment
10 first clothing fabric
10E end portion
11 first coupling member
12 second coupling member
14 electronic equipment connection portion
15 electrode
16 first insulating layer
17 conductive layer
18 second insulating layer
19 wiring
20 second clothing fabric
30 belt-like member
31 third coupling member
32 fourth coupling member
41, 42 system for managing a health condition of a calf
50 analysis apparatus
51 measurement unit
51a beat interval measuring unit
51b acceleration measuring unit
52 calculation unit
53 specification unit
54 determination unit
55 warning unit
56 thermoregulation unit
57 transmission and reception unit
58 storage unit
59 web server
60 measurement instrument
61 thermoregulator
62 warning apparatus
63 transceiver

1. A method for managing a health condition of a calf, comprising:
measuring beat intervals and acceleration associated with movement;
specifying a first resting period in which the acceleration is a constant M or less;
specifying a second resting period that is a period having a starting point at a time point after an end point of the first resting period and in which the acceleration is the constant M or less;
calculating at least one of an average value of beat intervals, a variation in beat intervals, a beat number, HF, or LF/HF in the first resting period;
calculating at least one of an average value of beat intervals, a variation in beat intervals, a beat number, HF, or LF/HF in the second resting period;
issuing a warning for notifying abnormality of a body temperature of the calf when a magnitude relationship of at least one of formulae (1) to (5) below is satisfied; and
raising or lowering the body temperature of the calf:
the average value of beat intervals in the first resting period>the average value of beat intervals in the second resting period (1)
the beat number in the first resting period<the beat number in the second resting period (2)
the variation in beat intervals in the first resting period>the variation in beat intervals in the second resting period (3)
the HF in the first resting period>the HF in the second resting period (4)
the LF/HF in the first resting period<the LF/HF in the second resting period (5)
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.

2. The method for managing a health condition of a calf according to claim 1, wherein the body temperature of the calf is raised by using a heat retaining garment, heating equipment, or a hot-water supply machine.

3. The method for managing a health condition of a calf according to claim 1, wherein the calf is cattle under nine months of age.

4. The method for managing a health condition of a calf according to claim 1, wherein each of the average values of beat intervals is an average value of RRIs each of which is an interval between an R wave and an R wave in an electrocardiographic signal.

5. The method for managing a health condition of a calf according to claim 1, wherein the beat number in the first resting period and the beat number in the second resting period are each a heart rate or a pulse rate.

6. The method for managing a health condition of a calf according to claim 1, wherein each of the variations in beat intervals is at least one of SDNN, RMSSD, CVRR, NN50, or pNN50,
provided that, the SDNN is a standard deviation of an RRI that is an interval between an R wave and an R wave in an electrocardiographic signal,
the RMSSD is a square root of an average value of squares of differences between consecutive adjacent RRIs,
the CVRR is a value obtained by dividing a value of the SDNN by an average value of RRIs and multiplying the obtained value by 100,
the NN50 is a value indicating a total number of times in which differences between the consecutive adjacent RRIs exceed 50 ms, and
the pNN50 is a value indicating a ratio of heartbeats in which the differences between the consecutive adjacent RRIs exceed 50 ms.

7. The method for managing a health condition of a calf according to claim 1, wherein the acceleration is expressed by a formula below:
Acceleration A=\sqrt{x²+y²+z²}−1 (I)
wherein x, y, and z are X-axis, Y-axis, and Z-axis acceleration obtained from an accelerometer attached to a body of the calf.

8. The method for managing a health condition of a calf according to claim 1, wherein the beat intervals are measured using a biological information measuring garment.

9. A system for managing a health condition of a calf, comprising:
a measurement unit that measures beat intervals and acceleration associated with movement;
a specification unit that specifies a first resting period in which the acceleration is a constant M or less, and specifies a second resting period that is a period having a starting point at a time point after an end point of the first resting period and in which the acceleration is the constant M or less;
a calculation unit that calculates at least one of an average value of beat intervals, a variation in beat intervals, a beat number, HF, or LF/HF in the first resting period, and calculates at least one of an average value of beat intervals, a variation in beat intervals, a beat number, HF, or LF/HF in the second resting period;
a determination unit that generates a warning signal when a magnitude relationship of at least one of formulae (1) to (5) below is satisfied; and
a warning unit that issues a waring for notifying abnormality of a body temperature of the calf from the warning signal:
the average value of beat intervals in the first resting period>the average value of beat intervals in the second resting period (1)
the beat number in the first resting period<the beat number in the second resting period (2)
the variation in beat intervals in the first resting period>the variation in beat intervals in the second resting period (3)
the HF in the first resting period>the HF in the second resting period (4)
the LF/HF in the first resting period<the LF/HF in the second resting period (5)
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.

10. The system for managing a health condition of a calf according to claim 9, comprising a thermoregulation unit that raises or lowers the body temperature of the calf.

11. The system for managing a health condition of a calf according to claim 9, wherein each of the average values of beat intervals is an average value of RRIs each of which is an interval between an R wave and an R wave in an electrocardiographic signal.

12. The system for managing a health condition of a calf according to claim 9, wherein the beat number in the first resting period and the beat number in the second resting period are each a heart rate or a pulse rate.

13. The system for managing a health condition of a calf according to claim 9, wherein each of the variations in beat intervals is at least one of SDNN, RMSSD, CVRR, NN50, or pNN50,
provided that, the SDNN is a standard deviation of an RRI that is an interval between an R wave and an R wave in an electrocardiographic signal,
the RMSSD is a square root of an average value of squares of differences between consecutive adjacent RRIs,
the CVRR is a value obtained by dividing a value of the SDNN by an average value of RRIs and multiplying the obtained value by 100,
the NN50 is a value indicating a total number of times in which the differences between the consecutive adjacent RRIs exceed 50 ms, and
the pNN50 is a value indicating a ratio of heartbeats in which the differences between the consecutive adjacent RRIs exceed 50 ms.

14. The system for managing a health condition of a calf according to claim 9, wherein the acceleration is expressed by a formula below:
Acceleration A=\sqrt{x²+y²+z²}−1 (I)
wherein x, y, and z are X-axis, Y-axis, and Z-axis acceleration obtained from an accelerometer attached to a body of the calf.

15. A garment comprising at least a part of the system for managing a health condition of a calf according to claim 9.

16. An apparatus for managing a health condition of a calf, comprising:
a measurement unit that measures beat intervals and acceleration associated with movement;
a specification unit that specifies a first resting period in which the acceleration is a constant M or less, and specifies a second resting period that is a period having a starting point at a time point after an end point of the first resting period and in which the acceleration is the constant M or less;
a calculation unit that calculates at least one of an average value of beat intervals, a variation in beat intervals, a beat number, HF, or LF/HF in the first resting period, and calculates at least one of an average value of beat intervals, a variation in beat intervals, a beat number, HF, or LF/HF in the second resting period; and
a determination unit that generates a warning signal when a magnitude relationship of at least one of formulae (1) to (5) below is satisfied:
the average value of beat intervals in the first resting period>the average value of beat intervals in the second resting period (1)
the beat number in the first resting period<the beat number in the second resting period (2)
the variation in beat intervals in the first resting period>the variation in beat intervals in the second resting period (3)
the HF in the first resting period>the HF in the second resting period (4)
the LF/HF in the first resting period<the LF/HF in the second resting period (5)
provided that, each LF is a value obtained by performing definite integration of a power spectrum from frequencies Lf1 to Lf2, the power spectrum being obtained by including a step of performing frequency spectrum conversion on beat intervals, and each HF is a value obtained by performing definite integration of the power spectrum from frequencies Hf1 to Hf2, where Hf1>Lf1 and Hf2>Lf2 are satisfied.
